(19) United States
(12) Patent Application Publication
(10) Pub. No.: US 2011/0301639 A1
(43) Pub. Date: Dec. 8, 2011

(54) One-Part Moisture-Curable Tissue Sealant

(76) Inventor: Eric J. Beckman, Aspinwall, PA (US)

(21) Appl. No.: 13/116,571

(22) Filed: May 26, 2011

Related U.S. Application Data
(60) Provisional application No. 61/349,274, filed on May 28, 2010.
(60) Provisional application No. 61/388,321, filed on Sep. 30, 2010.

(51) Int. Cl. A61B 17/03; A61L 15/26; C08G 65/48; C08G 77/18; C08G 77/26

(52) U.S. Cl. 606/213; 528/29; 528/28; 525/50; 528/26; 523/118

(57) ABSTRACT

A tissue sealant is described that includes the reaction product of (a) a polyol having at least two groups capable of reacting with an alkoxy silane and (b) an alkoxy silane having the formula: (R¹R²R³)—Si—CH₂—Z where (i) Z is an —OH, —SH, —NCO, or —NHR⁴ group, where R⁴ is hydrogen or an alkyl group; and (ii) each R¹, R², and R³, independently, is H, an alkoxy group, an alkyl group, a heteroalkyl group other than an alkoxy group, an aryl group, or a heteroaryl group, with the proviso that at least two of R¹, R², and R³ are alkoxy groups. The tissue sealant is moisture-curable and biodegradable in a physiological environment.

### CLAIM OF PRIORITY

This application claims the benefit of priority under 35 USC §119(e))(1) to U.S. Provisional application Ser. No. 61/349,274 filed May 28, 2010 and U.S. Provisional application Ser. No. 61/388,321 filed on Sep. 30, 2010, the entire contents of which are hereby incorporated by reference.

### TECHNICAL FIELD

This invention relates to moisture-curable sealants for sealing biological tissue.

### BACKGROUND

Tissue sealants are typically used to stop bleeding during vascular or liver surgery, eliminate air leaks in the lungs, and to prevent adhesions. Examples of sealants used for this purpose include fibrin products, polyethylene glycol products, and albumin-based products. In each case, the tissue sealant consists of two distinct components that are mixed together just prior to application to tissue to cause a rapid, irreversible chemical reaction. This reaction transforms the mixture from a low viscosity liquid into an elastic solid that coats the target tissue. The sealants are designed to degrade within a set period of time that typically ranges from days to weeks. One problem with such two-part sealants, however, is that the rapid cure times can cause the sealant applicator to clog.

### SUMMARY

A tissue sealant is described that includes the reaction product of (a) a polyol having at least two groups capable of reacting with an alkoxy silane; and (b) an alkoxy silane having the formula: (R¹R²R³)—Si—CH₂—Z where (i) Z is an —OH, —SH, —NCO, or —NHR⁴ group, where R⁴ is hydrogen or an alkyl group; and (ii) each R¹, R², and R³, independently, is H, an alkoxy group, an alkyl group, a heteroalkyl group other than an alkoxy group, an aryl group, or a heteroaryl group, with the proviso that at least two of R¹, R², and R³ are alkoxy groups. The tissue sealant is moisture-curable and biodegradable in a physiological environment.

As used herein, the term “alkyl” includes straight chain, branched, and cyclic alkyl groups.

The polyol may be an activated polyol. As used herein, the term “activated polyol” refers to a polyol in which one or more of the hydroxyl groups have been modified to create a molecule that is more reactive towards the alkoxy silane than the unmodified polyol.

In some embodiments, the activated polyol is the reaction product of a polyol and a polyfunctional linker molecule having at least one functional group capable of reacting with a hydroxyl group of the polyol, and at least one functional group capable of reacting with the Z group of the alkoxy silane. The functional group of the polyfunctional linker molecule that reacts with the hydroxyl group of the polyol can be different from, or the same as, the functional group of the polyfunctional linker molecule that reacts with the Z group of the alkoxy silane.

In some embodiments, the polyfunctional linker molecule is a polyisocyanate. Examples of suitable polyisocyanates include polyisocyanates derived from amino acids and amino acid derivatives such as lysine diisocyanate and derivatives thereof, lysine triisocyanate and derivatives thereof, and combinations thereof. Examples of suitable derivatives include alkyl esters (e.g., methyl and ethyl esters). Dipeptide derivatives can also be used. For example, lysine can be combined in a dipeptide with another amino acid (e.g., valine or glycine). Another representative example of a suitable polyfunctional linker molecule is maleic anhydride.

In some embodiments, the activated polyol includes an ester group capable of reacting with the alkoxy silane. The ester group may be created by reacting a hydroxyl group of a polyol with an anhydride to convert the hydroxyl group to a carboxylic acid group, and then reacting the carboxylic acid group with a reagent such as N-hydroxysuccinimide to create the ester group.

In some embodiments, the polyol is ionically charged. For example, the ionically charged polyol may include one or more sulfate, sulfonate, and/or ammonium ion functional groups.

In some embodiments, the reaction product of the polyol and alkoxy silane includes at least one hydrolyzable linkage. Examples of hydrolyzable linkages include esters, amides, urethanes, ureas, carbonates, and combinations thereof.

The alkoxy group of the alkoxysilane may be a C₁-C₆ alkoxy group, e.g., an ethoxy group. In some embodiments, two of R¹, R², and R³ are alkoxy groups, while in other embodiments each of R¹, R², and R³ is an alkoxy group. In some embodiments, the Z group of the alkoxysilane is an —NHR⁴ group.

In some embodiments, the polyol has a molecular weight that is no greater than 10,000, while in other embodiments it has a molecular weight that is no greater than 5,000. Representative examples include polyether polyols, polyester polyols, co-polyester polyether polyols, and combinations thereof. Two or more different polyols may be used in combination with each other and reacted with the alkoxysilane. As used herein, “different” means different molecular weights and/or chemical structures.

The tissue sealant can also include at least one reagent selected from the group consisting of solvents, diluents, catalysts, and combinations thereof. In use, the sealant is applied to a tissue surface, and cured in the presence of moisture associated with the tissue to seal the tissue surface. Because the sealant is a one-component composition (i.e. it includes one active molecule that moisture cures upon application to tissue), it is not necessary to mix two components prior to tissue application, thereby simplifying application from the user's perspective and avoiding the applicator clogging problems associated with two-component tissue sealants.

The details of one or more embodiments of the invention are set forth in the description below. Other features, objects, and advantages of the invention will be apparent from the description and from the claims.

### DETAILED DESCRIPTION

The tissue sealant includes the reaction product of a polyol and an alkoxy silane. The reaction product preferably includes at least one hydrolyzable linkage to promote biodegradability in vivo. Examples of hydrolyzable linkages include esters, amides, urethanes, ureas, carbonates, and combinations thereof.

The polyol may be an activated polyol in which one or more of the hydroxyl groups have been modified to create a molecule that is more reactive towards the alkoxy silane than the unmodified polyol. The activated polyol may be prepared by reacting a hydroxyl group of the polyol with a polyfunctional linker molecule having at least one functional group capable of reacting with a hydroxyl group of the polyol, and at least one functional group capable of reacting with the Z group of the alkoxy silane. Examples of suitable polyfunctional linker molecules are described in the Summary of the Invention, above. Alternatively, the activated polyol may be prepared by reacting a hydroxyl group of a polyol with an anhydride to convert the hydroxyl group to a carboxylic acid group, and then reacting the carboxylic acid group with a reagent such as N-hydroxysuccinimide to create an ester group. The ester group, in turn, is capable of reacting with the alkoxy silane.

Examples of suitable polyols are described in the Summary of the Invention, above. Specific examples of polyether polyols include polyethylene and polypropylene glycols. One or more of the hydroxyl groups may be activated. Specific examples of polyester polyols include polycaprolactone and polylactide diols. One or more of the hydroxyl groups may be activated.

The alkoxy silane has the formula: (R¹R²R³)—Si—CH₂—Z where (i) Z is an —OH, —SH, —NCO, or —NHR⁴ group. R⁴ is a hydrogen or an alkyl group (e.g., a C₁-C₆ alkyl group). Each R¹, R², and R³, independently, is H, an alkoxy group (e.g., a C₁-C₆ alkoxy group), an alkyl group (e.g., a C₁-C₆ alkyl group), a heteroalkyl group other than an alkoxy group (e.g., an alkyl amido or amido group), an aryl group (e.g., a phenyl group), or a heteroaryl group (e.g., a pyrrolyl, furyl, or pyridinyl group), with the proviso that at least two of R¹, R², and R³ are alkoxy groups. The alkyl groups may be straight chain, branched, or cyclic alkyl groups.

In one embodiment, the polyol is a polyalkylene glycol such as polyethylene glycol; the alkoxy silane is a trialkoxy silane such as a triethoxy silane in which the Z group is an alkylamino group such as a cyclohexylamino group (where R⁴ is a cyclohexyl group); and the polyfunctional linker molecule is a multi-functional isocyanate such as lysine diisocyanate (“LDI”) or a derivative thereof (e.g., alkyl esters such as methyl or ethyl esters), or lysine triisocyanate (“LTI”) or a derivative thereof (e.g., alkyl esters such as methyl or ethyl esters). The linker molecule reacts with the hydroxyl groups of the polyol to create an activated polyol.

In another embodiment, the polyol is a polyalkylene glycol such as polyethylene glycol in which one or more hydroxyl groups have been converted to activated ester groups, and the alkoxy silane is a trialkoxy silane such as a triethoxy silane in which the Z group is an alkylamino group such as a cyclohexylamino group (where R⁴ is a cyclohexyl group).

The sealants may further contain one or more reagents selected from the group consisting of solvents, diluents, catalysts, and combinations thereof. The reagents preferably are inert towards the polyol, trialkoxy silane, and polyfunctional linker molecule, and thus do not interfere with the reaction among these three reactants.

Examples of suitable catalysts, include tertiary amines (e.g., aliphatic tertiary amines) and organometallic compounds (e.g., bismuth salts and zirconium chelates). When the reactants include polyols and polyisocyanates, specific examples of useful catalysts include 1,4-diazabicyclo[2.2.2]octane (“DABCO”), 2,2′-dimorpholine diethyl ether (“DMDEE”), dibutyltin dilaurate (“DBTDL”), bismuth-2-ethylhexanoate, and combinations thereof.

The solvents and diluents may be used to modify the rheology of the sealant. Examples of suitable solvents include dimethylsulfoxide (DMSO), dimethylformamide (DMF), tetrahydrofuran (THF), glyme, and combinations thereof. Examples of suitable non-volatile diluents include dimethylsulfoxide (DMSO), propylene carbonate, diglyme, polyethylene glycol diacetates, polyethylene glycol dicarbonates, dimethylisosorbide, ethyl pyruvate, triacetin, triethylene glycol, and combinations thereof. Examples of suitable volatile diluents include hydrocarbons, perfluoroalkanes, hydrofluoroalkanes, carbon dioxide, and combinations thereof. A single reagent can perform multiple roles. Thus, for example, DMSO can function as both a solvent and a non-volatile diluent.

The sealants may also include one or more stabilizers. Examples include antioxidants (e.g., BHT and BHA), water scavengers (e.g., acyl and aryl halides, and anhydrides), Bronsted acids, and the like. Bronsted acids may also be used as catalysts.

The sealants may be prepared in either a single step reaction, in which reactants are combined together in a “single pot” reaction, or a multi-step reaction, in which the reactants are reacted sequentially. In either case, the reaction may be carried out in the presence of the aforementioned solvents, diluents, and/or stabilizers; alternatively, any or all of these reagents can be added after the reaction product has been created.

### EXAMPLE 1

Polyethylene glycol having a molecular weight ranging from 300 to 1500 is dissolved in an inert diluent. The amount of the diluent typically represents 40-60% by weight of the total reaction mixture. Lysine ethyl ester diisocyanate (“LDI”) is added at a 2:1 molar ratio relative to the glycol. A bismuth catalyst is added as well, and the glycol and diisocyanate are allowed to react with each other for several hours to create a polyurethane have isocyanate groups available for further reaction. Next, cyclohexylaminomethyl triethoxysilane is added at a 2:1 molar ratio relative to the glycol. The silane then reacts with the unreacted isocyanate groups to form the final moisture-curable product. Upon application to biological tissue, the product crosslinks in the presence of moisture associated with the tissue to form a smooth, elastomeric coating on the tissue that seals the tissue.

### EXAMPLE 2

In a typical example, polyethylene glycol (molecular weight either 600, 1500, or 3400) is mixed with glutaric anhydride (2 moles anhydride per mole of polyethylene glycol); the mixture is heated to 70-80° C. and allowed to stir overnight. IR shows the absence of anhydride peaks (1765, 1809 cm⁻¹) and the appearance of peaks representing the ester (1734 cm⁻¹) and carboxylic acid (1719 cm⁻¹). This intermediate is known as PEG-(COOH)₂.

PEG-(COOH)₂ is dissolved in anhydrous acetonitrile. N-hydroxy succinimide (2.2 moles per mole of PEG-(COOH)₂) is added and the mixture stirred until one phase is obtained. This solution is then cooled to <10° C. using an ice bath. At this point, dicylohexyl carbodiimide (2.2 moles per mole of PEG-(COOH)₂) in acetonitrile is added dropwise; after the addition is complete the mixture is stirred in the ice bath for 1 hour (a white precipitate begins to form). The ice bath is then removed and the mixture stirred at room temperature overnight. The precipitate (dicylohexyl urea) is removed by filtration. The filtrate is concentrated under vacuum, and the product precipitated into hexane. The product is redissolved in THF, filtered, then concentrated under vacuum. IR shows the characteristic NHS ester peaks at (1742, 1787, and 1815 cm⁻¹). This intermediate is known as PEG-(COONHS)₂.

PEG-(COOHNHS)₂ is dissolved in THF and heated to 50° C. At this point cyclohexylaminomethyl triethoxysilane (2 moles per mole of PEG-(COONHS)₂) is added and the mixture allowed to stir at 50° C. for 24 hours. The solution is cooled, filtered, then the THF is removed under vacuum. IR shows the formation of the amide at 1678 cm⁻¹. A sealant is created by mixing this product with various amounts of ethyl pyruvate.

A number of embodiments of the invention have been described. Nevertheless, it will be understood that various modifications may be made without departing from the spirit and scope of the invention. Accordingly, other embodiments are within the scope of the following claims.

What is claimed is:

1. A tissue sealant comprising the reaction product of:
(a) a polyol having at least two groups capable of reacting with an alkoxy silane; and
(b) an alkoxy silane having the formula: (R¹R²R³)—Si—CH₂—Z where:
(i) Z is an —OH, —SH, —NCO, or —NHR⁴ group, where R⁴ is hydrogen or an alkyl group; and
(ii) each R¹, R², and R³, independently, is H, an alkoxy group, an alkyl group, a heteroalkyl group other than an alkoxy group, an aryl group, or a heteroaryl group, with the proviso that at least two of R¹, R², and R³ are alkoxy groups, wherein the tissue sealant is moisture-curable and biodegradable in a physiological environment.

2. A tissue sealant according to claim 1 wherein the reaction product comprises at least one hydrolysable linkage.

3. A tissue sealant according to claim 2 wherein the hydrolyzable linkage is selected from the group consisting of esters, amides, urethanes, ureas, carbonates, and combinations thereof.

4. A tissue sealant according to claim 1 wherein the alkoxy group is a C₁-C₆ alkoxy group.

5. A tissue sealant according to claim 1 wherein the alkoxy group is an ethoxy group.

6. A tissue sealant according to claim 1 wherein two of R¹, R², and R³ are alkoxy groups.

7. A tissue sealant according to claim 1 wherein each of R¹, R², and R³ is an alkoxy group.

8. A tissue sealant according to claim 1 wherein Z is an —NHR⁴ group.

9. A tissue sealant according to claim 1 wherein the polyol has a molecular weight that is no greater than 10,000.

10. A tissue sealant according to claim 1 wherein the polyol has a molecular weight that is no greater than 5,000.

11. A tissue sealant according to claim 1 wherein the polyol is selected from the group consisting of polyether polyols, polyester polyols, co-polyester polyether polyols, and combinations thereof

12. A tissue sealant according to claim 1 wherein the polyol is an activated polyol.

13. A tissue sealant according to claim 12 wherein the activated polyol is prepared by reacting one or more hydroxyl groups with a polyfunctional linker molecule having at least one functional group capable of reacting with the hydroxyl groups of the polyol, and at least one functional group capable of reacting with the Z group of the alkoxy silane.

14. A tissue sealant according to claim 13 wherein the functional group of the polyfunctional linker molecule that reacts with the hydroxyl group of the polyol to form the activated polyol is different from the functional group of the polyfunctional linker molecule that reacts with the Z group of the alkoxy silane.

15. A tissue sealant according to claim 13 wherein the functional group of the polyfunctional linker molecule that reacts with the hydroxyl group of the polyol to form the activated polyol is the same as the functional group of the polyfunctional linker molecule that reacts with the Z group of the alkoxy silane.

16. A tissue sealant according to claim 13 wherein the polyfunctional linker molecule comprises a polyisocyanate.

17. A tissue sealant according to claim 16 wherein the polyisocyanate is selected from the group consisting of lysine diisocyanate and derivatives thereof, lysine triisocyanate and derivatives thereof, and combinations thereof.

18. A tissue sealant according to claim 13 wherein the polyfunctional linker molecule comprises maleic anhydride.

19. A tissue sealant according to claim 12 wherein the activated polyol comprises one or more ester groups.

20. A tissue sealant according to claim 19 wherein the activated polyol is prepared by converting one or more hydroxyl groups of a polyol to carboxylic acid groups, and esterifying the carboxylic acid groups to create an activated polyol comprising one or more ester groups.

21. A tissue sealant according to claim 1 wherein the tissue sealant comprises the reaction product of the alkoxy silane and at least two different polyols.

22. A tissue sealant according to claim 1 wherein the polyol is ionically charged.

23. A tissue sealant according to claim 22 wherein the polyol includes at least one functional group selected from the group consisting of sulfates, sulfonate, ammonium ions, and combinations thereof.

24. A tissue sealant according to claim 1, wherein the tissue sealant further comprises at least one reagent selected from the group consisting of solvents, diluents, catalysts, and combinations thereof.

25. A method of sealing tissue comprising:
(A) applying a sealant according to claim 1 to a tissue surface; and
(B) curing the sealant to seal the area of tissue to which the sealant was applied.
